(19) United States
(12) Patent Application Publication
(10) Pub. No.: US 2010/0316735 A1
(43) Pub. Date: Dec. 16, 2010

(54) COMPOSITIONS AND METHODS FOR REDUCING HANGOVER SYMPTOMS

(76) Inventor: Wade Belliston, Los Angeles, CA (US)

Correspondence Address: David B. Tingey, 1800 Eagle Gate Tower, 60 East South Temple, Salt Lake City, UT, 84145, US

(21) Appl. No.: 12/757,856

(22) Filed: Apr. 9, 2010

Related U.S. Application Data
(60) Provisional application No. 61/168,420, filed on Apr. 10, 2009.

(51) Int. Cl. A61K 33/06; A61K 36/33; A61K 36/82; A61K 33/00; A61P 43/00

(52) U.S. Cl. 424/682; 424/767; 424/729; 424/722

(57) ABSTRACT

Compositions and methods for reducing hangover symptoms. Generally, a hangover reducing composition includes one or more plant extracts, such as prickly pear cactus extract, white willow bark extract, milk thistle extract, green tea extract, and combinations thereof. In some cases, the composition further includes supplemental nutrients, such as vitamins and minerals (including electrolytes). Some examples of suitable nutrients include sodium, potassium, magnesium, and vitamins C, B1, B2, B3, B6, and B12. The composition is administered to a person in any suitable form, including as a carbonated soft drink, an energy drink, a tea, a sports drink, a non-carbonated drink, a pill, an elixir, and so forth. In some preferred instances, the composition is formulated as a carbonated soft drink. In order to reduce hangover symptoms, the composition is ingested before, during, and/or after the consumption of a drug, such as alcohol.

### CROSS-REFERENCE TO RELATED APPLICATION

This application claims priority to U.S. Provisional Patent Application Ser. No. 61/168,420, filed Apr. 10, 2009, entitled COMPOSITIONS AND METHODS FOR REDUCING HANGOVER SYMPTOMS, which is incorporated herein in its entirety by reference.

### BACKGROUND OF THE INVENTION

1. Field of the Invention

The present invention relates to compositions and methods for reducing the symptoms of a hangover. In particular, the present invention relates to compositions and methods for reducing the symptoms and effects of a hangover through the administration of a hangover reducing composition containing one or more plant extracts that counteract the hangover symptoms.

2. Background and Related Art

The term “hangover” is often used to describe the unpleasant physiological effects that follow the heavy consumption of a drug, such as alcohol. Although the severity and the specific symptoms of a hangover often vary from one person to another and from one hangover to another, some common symptoms of a hangover include headache, nausea, vomiting, sensitivity to light and noise, fatigue, dehydration, diarrhea, flatulence, elevated body temperature, hypersalivation, irritability, anxiety, difficulty concentrating, trouble sleeping, lethargy, lack of depth perception, thirst, and other disagreeable sensations. Additionally, while the symptoms of a hangover may last a few hours, in some cases, these symptoms can last as long as two or three days after the alcohol was last consumed.

Currently, people attempt to treat and reduce hangover symptoms in variety ways. In some instances, people take acetylsalicylic acid (e.g., aspirin) or acetaminophen to relieve the pain associated with a hangover. In other instances, people try to shorten a hangover's duration by drinking water to cleanse their system and replenish moisture that was lost due to the increased urine production caused by alcohol. In yet other instances, people exercise in order to increase their heart rate and blood oxygen levels, and thereby increase the rate at which their body metabolizes the consumed alcohol. In still other instances, people ingest chemicals, such as activated carbon, baking soda, coffee, caffeinated chasers (e.g., caffeinated energy drinks), or other substances that are believed to shorten the duration of a hangover or to minimize hangover symptoms.

Nevertheless, many of the techniques currently used to alleviate the symptoms associated with a hangover have shortcomings. For instance, because acetylsalicylic acid is highly acidic, it tends to cause stomach irritation, and can even cause stomach bleeding, when taken with alcohol. Similarly, when acetaminophen is mixed with alcohol, the combination can have toxic results on a liver. Furthermore, some techniques, such as exercising, drinking a lot of water, or drinking a caffeinated drink can also be inconvenient to a person suffering from a hangover, especially when the person wants to sleep. Additionally, while some conventional techniques work to relieve hangover symptoms, at least to a limited extent, other techniques may have no effect, besides a placebo effect, on hangover symptoms.

Thus, while techniques currently exist that are used to reduce hangover symptoms, challenges still exist, including those listed above. Accordingly, it would be an improvement in the art to augment or even replace current techniques with other techniques.

### SUMMARY OF THE INVENTION

The present invention relates to compositions and methods for reducing the symptoms of a hangover. In particular, the present invention relates to compositions and methods for reducing the symptoms and effects of a hangover through the administration of a hangover reducing composition containing one or more ingredients that counteract hangover symptoms.

Implementation of the present invention takes place in association with a hangover reducing composition that comprises one or more plant extracts that are capable of reducing hangover symptoms when ingested. The composition can comprise any suitable plant extract or combination of extracts that allows the beverage to reduce the uncomfortable sensations that are associated with a hangover. Some examples of such plant extracts include prickly pear cactus extract, white willow bark extract, milk thistle extract, and green tea extract.

In addition to plant extracts, the composition can further comprise any other suitable ingredient that allows the composition to perform its intended purpose of reducing hangover symptoms. In some implementations, for instance, the composition comprises one or more vitamins and minerals (e.g., sodium, potassium, magnesium, vitamin C, vitamin B1, vitamin B2, vitamin B3, vitamin B6, vitamin B12, and combinations thereof), sweeteners (e.g., sugar, high fructose corn syrup, sugar substitutes, etc.), flavorings (e.g., orange, cranberry, mangosteen, passion fruit, and other suitable flavorings), preservatives (e.g., sodium benzoate), carbonation, and/or water.

While the described compositions and methods of the present invention have proven to be particularly useful for the treatment of hangover symptoms stemming from the consumption of alcohol, the skilled artisan will appreciate that the described composition can be used to treat hangover symptoms or hangover-like symptoms caused by a variety of other factors. For example, the composition can be used to treat hangover-like symptoms stemming from allergies, sickness, the use of anesthesia, and so forth.

Additionally, while in some presently preferred forms, the composition is formulated as a carbonated beverage, the composition can be formulated in a variety of different forms, including as an energy drink, a sports drink, a tea, a non-carbonated drink, and any other suitable form. However, it should be noted that the composition need not be produced as a beverage. Instead, the composition can be produced in any other suitable form, including as a pill, a tablet, a capsule, a chewable formulation, an elixir, a suspension, an emulsion, a syrup, a food additive, a powdered drink mix, and other forms for ingestion, consumption or administration.

These and other features and advantages of the present invention will be set forth or will become more fully apparent in the description that follows and in the appended claims. The features and advantages may be realized and obtained by means of the instruments and combinations particularly pointed out in the appended claims. Furthermore, the features and advantages of the invention may be learned by the practice of the invention or will be obvious from the description, as set forth hereinafter.

### DETAILED DESCRIPTION OF THE INVENTION

The present invention relates to compositions and methods for reducing the symptoms of a hangover. In particular, the present invention relates to compositions and methods for reducing the symptoms and effects of a hangover through the administration of a hangover reducing composition containing one or more plant extracts that counteract the hangover symptoms.

Generally, the described hangover reducing composition is formulated to reduce hangover symptoms after the composition has been ingested. It should be noted that in the disclosure and in the claims, the term “reduce” and variations thereof, may refer to the reduction, relief, prevention, and/or alleviation of one or more symptoms associated with a hangover or of a hangover-like symptom.

The hangover reducing composition can have any suitable form that allows it to be ingested, consumed or administered. In at least some embodiments, the hangover reducing composition can have is orally ingested. Indeed, embodiments of the present invention embrace the composition as a beverage, a tablet, a capsule, a pill, a chewable solid formulation, a food additive, a suspension, an elixir, a syrup, an emulsion, a powdered drink mix, or another suitable form. In some embodiments, the composition comprises a beverage. While the following discussion focuses on the beverage form of the composition, the skilled artisan will recognize that the composition may be formulated in any other suitable form through the addition of a suitable carrier, diluent, excipient, and/or other suitable substance.

The hangover reducing composition can comprise any suitable ingredient that allows it to reduce hangover symptoms after the composition has been ingested. In some preferred embodiments, however, the composition includes one or more plant extracts, supplemental nutrients, flavoring agents, sweeteners, and/or other suitable substances. To provide a better understanding of the hangover reducing composition, each of the aforementioned ingredients is described below in more detail.

In some embodiments, the described composition comprises one or more plant extracts that reduce the undesirable symptoms of a hangover. While the composition can comprise any suitable plant extract, some examples of suitable plant extracts include prickly pear cactus extract, white willow bark extract, milk thistle extract, and green tea extract (i.e., caffeinated or decaffeinated tea extract). Additionally, although the composition can comprise any suitable combination of plant extracts, in some presently preferred embodiments, the composition comprises prickly pear cactus extract, white willow bark extract, and milk thistle extract. In other embodiments, the composition comprises prickly pear cactus extract, white willow bark extract, milk thistle extract, and green tea extract (e.g., decaffeinated).

The plant extracts in the composition can serve a variety of purposes. In one example, it is believed that the prickly pear extract acts to support liver function and to reduce body pains (e.g., headaches). In another example, it is believed that the white willow bark extract acts as a natural analgesic that reduces pains (e.g., headache) associated with a hangover. In yet another example, it is believed that milk thistle extract helps to support liver function to speed the metabolization of alcohol. In a final example, it is believed that the green tea extract acts a strong antioxidant that combats the oxidizing effects that alcohol has on chemicals within the body.

In some embodiments, the composition comprises one or more supplemental nutrients. As used herein, the term “supplemental nutrient” and variations thereof, may refer to a vitamin, mineral (i.e., an electrolyte), and/or other suitable substance (in addition to the aforementioned plant extracts and the sweeteners discussed below) that is capable of providing nourishment to the body.

These supplement nutrients can help reduce the symptoms of a hangover in several ways. By way of example, the supplemental nutrients can help replenish nutrients that are lost or broken down due to heavy alcohol consumption. For instance, where alcohol breaks down nutrients in the body or causes them to be excreted, the consumption of alcohol can cause nutrient deficiencies that lead to hangover symptoms (e.g., nausea, sensitivity to light and noise, etc.). Accordingly, when a person ingests the composition containing supplemental nutrients, the supplemental nutrients can help to replenish the person's nutrient levels so as reduce hangover symptoms associated with the alcohol-related nutrient deficiencies.

Where the composition comprises one or more supplemental nutrients, the composition can comprise any vitamin, mineral (i.e., electrolyte), or other nourishing substance that is safe for consumption and which allows the composition to perform its intended purposes. Some examples of suitable nutrients comprise vitamin C, vitamin B1, vitamin B2, vitamin B3, vitamin B5, vitamin B6, vitamin B7, vitamin B9, vitamin B12, vitamin A, vitamin D, vitamin E, vitamin K, potassium, sodium, magnesium, zinc, calcium, and iron. In some preferred embodiments, however, the composition comprises one or more of the following nutrients: vitamin C, vitamin B1, vitamin B2, vitamin B3, vitamin B6, vitamin B12, sodium, potassium, and magnesium.

In some embodiments, the composition also comprises one or more flavoring agents that provide the composition with a desired taste. While the composition can comprise virtually any suitable flavoring agent (e.g., a root-beer, cherry, lemon, grape, apple, peach, berry, or other flavoring agent), in some preferred embodiments, the composition comprises a flavoring agent selected from a cranberry flavoring agent, a citrus (e.g., orange) flavoring agent, a mangosteen flavoring agent, a passion fruit flavoring agent, and combinations thereof. Indeed, in some embodiments, the composition comprises a combination of cranberry and orange flavoring agents or a combination of mangosteen and passion fruit flavoring agents.

In some embodiments, the composition comprises one or more sweeteners. In addition to sweetening the composition, a sweetener can also serve other purposes, such as providing the composition with a desired texture, uniformity, and taste. Accordingly, the composition can comprise any suitable sweetener, or sweeteners, that provide the composition with a desired characteristic. In one example, the composition comprises one or more natural crystalline or liquid sweeteners, such as sucrose, glucose levulose, invert sugar, fructose, high fructose corn syrup, and other naturally occurring sweeteners. In another example, the composition comprises one or more artificial or non-nutritive sweeteners, such as saccharin, cyclamate, aspartame, other dipeptides, sucralose, neotame, acesulfame potassium, alitame, and the like. In some presently preferred embodiments, however, the sweetener in the composition comprises sucrose (sugar).

In addition to the aforementioned ingredients, the composition may comprise any other suitable ingredient. In one example, the composition comprises one or more preservatives. In this example, the composition can comprise any suitable preservative, including, but not limited to, potassium sorbate, sodium benzoate, and/or another edible preservative. In some presently preferred embodiments, the composition comprises sodium benzoate.

In another example of another suitable ingredient, the composition comprises water so that the composition can be ingested as a beverage. In this example, water acts as the vehicle or liquid portion in which the other ingredients of the composition are dissolved or dispersed. In order to not adversely affect the taste, odor, or appearance of the composition, in some preferred embodiments, the water comprises a filtered water that is substantially clear, colorless, low in alkalinity, and free from objectionable minerals, odors, tastes, and organic material.

In yet another example, where the composition comprises a beverage, the composition optionally comprises carbon dioxide. Carbon dioxide may provide the composition with several beneficial characteristics. For instance, carbon dioxide can enhance the flavor of the composition, provide the composition with effervescence, enhance the appearance of the composition, enhance the purity of the composition, and destroy bacteria in the composition by reducing the oxygen level in the composition.

While the composition may comprise any suitable amount of carbon dioxide, in some presently preferred embodiments, the composition comprises between about 0.5 and about 17 volumes of carbon dioxide. As used herein, one volume of carbon dioxide may refer to the amount of carbon dioxide that is absorbed by any given quantity of water at a temperature of 60 degrees Fahrenheit and at 1 atmosphere. Additionally, as used herein, one volume of gas occupies the same space as does the water by which it is absorbed.

In still another example of a suitable ingredient, in some embodiments in which the composition comprises a carbonated beverage, the composition further comprises one or more acids. In such embodiments, the acids may perform any suitable function, such providing tartness or acting as a mild preservative to the composition. Some examples of suitable acids that can be added to the composition include phosphoric, citric, malic, tartaric, lactic, formic, ascorbic, fumaric, and adipic acid.

In another still another example, where the composition is a beverage, the composition comprises caffeine, carbohydrates, and/or proteins. Accordingly, in this example, the composition can be formulated as an energy drink, a sports drink, and/or a protein drink, respectively.

In a final example, the composition can include any other ingredient that is suitable for consumption and that allows the composition to fulfill its intended purposes. Indeed, in some embodiments, the composition comprises one or more coloring agents, clouding agents, thickeners, and/or viscosity building agents (e.g., sodium methylcellulose and/or another gum).

In some embodiments, the composition is formulated to be administered to a person as a unit dose. In such embodiments, the various ingredients of the composition may be present in a single dose at any effective amount. In one embodiment, a dose of the composition comprises between about 12 milligrams (“mg”) and about 1.2 grams (“g”) of pure prickly pear cactus extract; between about 1 mg and about 100 mg of one or more of each of the following in pure form: green tea extract, white willow bark extract, and milk thistle; between about 5 mg and about 500 mg of one or more of each of the following: potassium, sodium, and vitamin C; and between about 0.01 mg and about 60 mg of one or more of each of the following: magnesium, vitamin B1, vitamin B2, vitamin B3, vitamin B6, and vitamin B12.

In another embodiment, a single dose of the composition comprises between about 80 mg and about 240 mg of pure prickly pear cactus extract; between about 5 mg and about 20 mg of one or more of each of the following in pure form: green tea extract, white willow bark extract, and milk thistle; between about 15 mg and about 140 mg of one or more of each of the following: potassium, sodium, and vitamin C; and between about 0.1 mg and about 12 mg of one or more of each of the following: magnesium, vitamin B1, vitamin B2, vitamin B3, vitamin B6, and vitamin B12.

In a presently preferred embodiment, a single dose of the composition comprises between about 110 mg and about 130 mg of pure prickly pear cactus extract; between about 6 mg and about 15 mg of one or more of each of the following in pure form: green tea extract, white willow bark extract, and milk thistle; between about 25 mg and about 80 mg of one or more of each of the following: potassium, sodium, and vitamin C; and between about 0.2 mg and about 8 mg of one or more of each of the following: magnesium, vitamin B1, vitamin B2, vitamin B3, vitamin B6, and vitamin B12.

The described composition can be used to reduce hangover symptoms in any suitable manner. In one example, the composition is taken before the consumption of alcohol. In another example, the composition is taken with alcohol. In still another example, the composition is taken at some period of time after the consumption of alcohol (e.g., as a chaser). In yet another example, the composition is taken as needed. For instance, a person can consume the composition before, during and/or after the consumption of alcohol. Indeed, the specific dose level (e.g., the amount of ingredients in the composition and the amount of the composition that a person ingests) and the frequency of consumption for any particular person may be varied and will depend upon a variety of factors, including the activity of the specific ingredients employed in the composition, the metabolic stability and length of action of those ingredients, the age, body weight, general health, sex, diet, mode and time of administration, rate of excretion, drug combination, the severity of the hangover, and so forth.

As discussed, the described composition can have many beneficial characteristics. In one example, the combination of the plant extracts allows the composition to reduce pain and other hangover symptoms while improving liver function. In another example, where the composition comprises one or more supplemental nutrients, the nutrients can replenish nutrients in the body that are lost or broken down as a result of alcohol consumption. By replenishing these nutrients, the composition helps the body return to its normal state of functioning and acts to reduce hangover symptoms. In some cases, the composition further acts to detoxify the body by removing harmful chemicals associated with alcohol. Additionally, because the composition is easily ingested (e.g., as a beverage or in another form (e.g., as a pill, elixir, etc.)), use of the composition can be convenient, especially where the composition has a pleasing taste. In another example of a beneficial characteristic, where the composition is caffeine free (e.g., comprises decaffeinated green tea extract), the composition, unlike some conventional chasers that comprise caffeine, may not impede sleep after being consumed. Furthermore, unlike acetylsalicylic acid and acetaminophen, the described composition is not damaging to the stomach or liver, even when taken with alcohol.

The composition may be made in any suitable manner. By way of example, where the composition comprises a carbonated soft drink, the composition can be produced by standard procedures, such as a cold filling or a hot filling technique. In another example, the composition is manufactured into a lyophilized powdered form that can be mixed with water to form a beverage. Accordingly, the composition can be manufactured as an instant drink mix. In still another example, the composition is manufactured as a dry powder that is packaged in a tea bag. In this example, the packet can be placed in hot water so that some or all of the bag's ingredients are dissolved into the water, allowing the composition to be consumed as a tea.

### Example

A single dose of a beverage form of the composition was prepared according to the following formula:

| INGREDIENT | AMOUNT PER DOSE | |
|---|---|---|
| Prickly Pear Cactus Extract (Powder) | 120 | mg |
| Decaffeinated Green Tea | 10 | mg |
| Extract (Powder) | | |
| White Willow Bark Extract (Powder) | 10 | mg |
| Milk Thistle Extract (Powder) | 10 | mg |
| Sodium | 70 | mg |
| Potassium | 30 | mg |
| Magnesium | 4 | mg |
| Vitamin C | 60 | mg |
| Vitamin B1 | 0.3 | mg |
| Vitamin B2 | 0.34 | mg |
| Vitamin B3 | 4 | mg |
| Vitamin B6 | 2 | mg |
| Vitamin B12 | 6 | mg |
| Sugar | 22 | grams |
| Citrus and Cranberry Flavoring agents | 780 | mg |
| Sodium Benzoate | 15 | mg |
| Carbonated and Filtered Water | Up to 8 | Oz. |

In this example, the various powder and/or liquid ingredients were mixed into the water and then carbonated to form a carbonated soft drink.

Thus, as discussed herein, the embodiments of the present invention embrace compositions and methods for reducing the symptoms of a hangover. In particular, the present invention relates to compositions and methods for reducing the symptoms and effects of a hangover through the administration of a hangover reducing composition containing one or more plant extracts that counteract the hangover symptoms.

The present invention may be embodied in other specific forms without departing from its spirit or essential characteristics. The described embodiments and examples are to be considered in every respect as illustrative only, and not as being limiting or restrictive. The scope of the invention is, therefore, indicated by the appended claims rather than by the foregoing description. All changes that come within the meaning and range of equivalency of the claims are to be embraced within their scope.

What is claimed is:

1. A composition for reducing hangover symptoms, comprising:
prickly pear cactus extract; and
a plant extract selected from the group consisting of white willow bark extract, milk thistle extract, green tea extract, and combinations thereof.

2. The composition of claim 1, wherein the composition comprises white willow bark extract, milk thistle extract, and green tea extract.

3. The composition of claim 1, wherein the composition further comprises a supplemental nutrient selected from the group consisting of sodium, potassium, magnesium, vitamin C, vitamin B1, vitamin B2, vitamin B3, vitamin B6, vitamin B12, and combinations thereof.

4. The composition of claim 1, wherein the composition comprises a beverage selected from a carbonated soft drink, an energy drink, a sports drink, a tea, a non-carbonated drink, and combinations thereof.

5. The composition of claim 1, wherein the composition comprises a carbonated soft drink.

6. The composition of claim 1, wherein the composition comprises an edible form selected from a pill, tablet, capsule, elixir, syrup, food additive, suspension, emulsion, powdered drink mix, and a chewable solid.

7. The composition of claim 1, wherein a dose of the composition comprises between about 80 mg and 240 mg of prickly pear cactus extract.

8. The composition of claim 7, wherein a dose of the composition comprises between about 5 mg and about 20 mg of a one or more the plant extracts selected from the group consisting white willow bark extract, milk thistle extract, green tea extract, and combinations thereof.

9. The composition of claim 1, wherein a dose of the composition comprises:
between about 80 mg and 240 mg of prickly pear cactus extract;
between about 5 mg and about 20 mg of a one or more the plant extracts selected from the group consisting white willow bark extract, milk thistle extract, green tea extract, and combinations thereof;
between about 15 mg and about 140 mg of a first supplemental nutrient selected from the group consisting potassium, sodium, vitamin C, and combinations thereof; and
between about 0.1 mg and about 12 mg of a second supplemental nutrient selected from the group consisting of magnesium, vitamin B1, vitamin B2, vitamin B3, vitamin B6, and vitamin B12.

10. A composition for reducing hangover symptoms, comprising:
prickly pear extract;
white willow bark extract;
milk thistle extract; and
a supplemental nutrient selected from the group consisting of sodium, potassium, magnesium, vitamin C, vitamin B1, vitamin B2, vitamin B3, vitamin B6, vitamin B12, and combinations thereof.

11. The composition of claim 10, further comprising decaffeinated green tea extract.

12. The composition of claim 10, wherein a dose of the composition comprises between about 80 mg and 240 mg of prickly pear cactus extract.

13. The composition of claim 10, wherein a dose of the composition comprises:
between about 80 mg and 240 mg of prickly pear cactus extract;
between about 5 mg and about 20 mg of a one or more the plant extracts selected from the group consisting white willow bark extract, milk thistle extract, green tea extract, and combinations thereof;
between about 15 mg and about 140 mg of a first supplemental nutrient selected from the group consisting potassium, sodium, vitamin C, and combinations thereof; and
between about 0.1 mg and about 12 mg of a second supplemental nutrient selected from the group consisting of magnesium, vitamin B1, vitamin B2, vitamin B3, vitamin B6, and vitamin B12.

14. The composition of claim 10, wherein the composition comprises a beverage selected from the group consisting of a carbonated soft drink, an energy drink, a sports drink, a tea, a non-carbonated drink, and combinations thereof.

15. The composition of claim 14, wherein the composition comprises a carbonated soft drink.

16. A method for reducing hangover symptoms, the method comprising:
consuming a drug; and
ingesting a hangover reducing composition comprising:
prickly pear cactus extract; and
a plant extract selected from the group consisting of white willow bark extract, milk thistle extract, green tea extract, and combinations thereof.

17. The method of claim 16, wherein the hangover reducing composition is ingested after the drug is consumed.

18. The method of claim 16, wherein the hangover reducing composition comprises white willow bark extract, milk thistle extract, and green tea extract.

19. The method of claim 16, wherein the hangover reducing composition further comprises a supplement selected from the group consisting of sodium, potassium, magnesium, vitamin C, vitamin B1, vitamin B2, vitamin B3, vitamin B6, vitamin B12, and combinations thereof.

20. The method of claim 16, wherein a dose of the composition comprises:
between about 80 mg and 240 mg of prickly pear cactus extract;
between about 5 mg and about 20 mg of a one or more the plant extracts selected from the group consisting white willow bark extract, milk thistle extract, green tea extract, and combinations thereof;
between about 15 mg and about 140 mg of a first supplemental nutrient selected from the group consisting potassium, sodium, vitamin C, and combinations thereof; and
between about 0.1 mg and about 12 mg of a second supplemental nutrient selected from the group consisting of magnesium, vitamin B1, vitamin B2, vitamin B3, vitamin B6, and vitamin B12.
